(19) United States
(12) Patent Application Publication
(10) Pub. No.: US 2025/0314035 A1
(43) Pub. Date: Oct. 9, 2025

(54) Pre-formed Vertical Access For Integrity Testing

(71) Applicant: A. H. Beck Foundation Co., Inc., Converse, TX (US)

(72) Inventors: August Henry Beck, IV, San Antonio, TX (US); August Henry Beck, III, Converse, TX (US)

(21) Appl. No.: 18/914,898

(22) Filed: Oct. 14, 2024

Related U.S. Application Data
(63) Continuation of application No. 18/625,892, filed on Apr. 3, 2024, now abandoned.

(51) Int. Cl. E02D 33/00; E02D 5/36; G01N 33/38

(52) U.S. Cl. CPC E02D 33/00; E02D 5/36; E02D 2250/0023; E02D 2300/002; E02D 2600/40; G01N 33/383

(57) ABSTRACT

A system and a method for providing preformed vertical access tunnels for testing foundation structures is described. The system comprises a structural cage having vertical structural members and confining structural members, and tubing running generally vertically and removably attached to the structural cage. The method of providing preformed vertical access tunnels for testing foundation structures comprises providing the system and an excavated hole, introducing the system into the excavated hole, pour concrete into the hole to encase structural cage, pressuring the removable tubing thereby expanding the diameter of the tubing, allow the concrete to cure while the tubing remains expanded by pressure, de-pressuring the tubing after the concrete has cured, and extracting the tubing thereby leaving generally vertical access tunnels in the foundation for testing.

### CROSS-REFERENCE TO RELATED APPLICATIONS

This application claims priority as a continuation of U.S. application Ser. No. 18/625,892. entitled Pre-Formed Vertical Access For Integrity Testing, which was filed Apr. 3, 2024. This priority application is incorporated herein by reference for all purposes including to describe the invention, the level of skill in the art, the background art, etc.

### STATEMENTS REGARDING FEDERALLY SPONSORED RESEARCH OR DEVELOPMENT

Not Applicable.

### REFERENCE TO A MICROFICHE APPENDIX

Not Applicable.

### BACKGROUND OF THE INVENTION

### 1. Field of the Invention

The present invention relates to a system and method for providing access for determining concrete integrity and continuity of structural objects, such as drilled and/or deep foundations. More particularly, the invention relates to a system and method for providing access for determining concrete integrity and continuity of deep foundations without permanently embedding tubes within the bored piles or other structural objects. More particularly, the present invention relates the invention relates to a system and method for providing access for determining concrete integrity and continuity of deep foundations utilizing removable tubing when forming the bored piles or other structural objects.

### 2. Description of the Related Art

Concrete integrity sensing and testing apparatuses have been used in the building industry for several years. These sensing apparatuses include a wide range of devices used for a wide range of reasons in the field. This includes sensing devices that are used in connection with the installation, testing, and use of supporting structures such as deep foundations that are used to support the weight of superstructures such as supporting the weight of buildings and bridges. As can be appreciated, it is important to both ensure that a supporting structure, such as a bored pile, has been properly installed and that it is in proper condition throughout its use in the field.

With respect to the installation of bored piles, it is important that these structures be properly constructed so that the bored pile can support the weight of a building or superstructure. Thus, over the years, systems have been designed to work in connection with the installation of a bored pile to ensure that this bored pile meets the building requirements for the structure. One example of the type of testing devices that have been utilized in connection with bored pile installations is shown in Piscsalko et al., U.S. Pat. No. 6,301,551. Furthermore, testing integrity does not necessarily end with the installation of a bored pile. Mcvay, et al., U.S. Pat. No. 6,533,502 discloses an example of a device used to monitor a bored pile after the installation process is completed. The information produced by the systems can be used to determine the current state of the bored pile and for determining damage that may or may not have incurred in response to any one of several events during construction and installation. One early detailed description of cast in-situ concrete bored pile testing is described in “Sonic Pulse Method of Testing Cast-In-Situ Concrete Piles” by J. F. Levy from the May 1970 issue of Ground Engineering (herein after “Levy article”). There are several methods of testing the integrity of poured structural objects. For example, Mullins, et al., U.S. Pat. No. 6,783,273 discloses an example method for testing the integrity of concrete shafts or bored piles. Furthermore, the ASTM D6760 provides guidelines on the use of sonic signal for integrity evaluations. U.S. Pat. Nos. 6,301,551, 6,533,502, 6,783,273 and ASTM D6760 and the Levy article are hereby incorporated by reference in their entirety for all purposes including background, level of skill in the art, and how to make and use the certain elements or steps of the claimed system and method.

One problem with the system and methods of the prior art systems is the cast-in-place longitudinal tubes (CSL) must run the entire length of the structure, in many cases hundreds of feet, for each testing access point in each bored pile. In total for a deep foundation project, the costs of the tubing can be very expensive. As can now be seen, there is a need for a system and a method that can provide convenient, stable access to foundation structures such as bored piles that does not require leaving unnecessary tubes in place in the structure.

### BRIEF SUMMARY OF THE INVENTION

The present invention is a system and a method for providing preformed vertical access tunnels for testing foundation structures. The system comprises a structural cage having vertical structural members as well as confining structural members, and tubing running generally vertically and removably attached to the structural cage. In preferred embodiments, the structural cage comprises a pre-formed cage with rebar forming the generally vertical structural members and the confining structural members being formed from rebar, preferably surrounding the vertical structural members, and being attached to the vertical structural members. The removal tubing is preferably a flexible hose capable of holding modest pressures that is removably attached to the structural cage and positioned generally parallel with the vertical structural members.

The method of providing preformed vertical access tunnels for testing foundation structures comprises providing the system and an excavated hole, introducing the system into the excavated hole, pour concrete into the hole to encase structural cage, pressuring the removable tubing during or after filling the hole with concrete thereby expanding the diameter of the tubing, allow the concrete to cure while the tubing remains expanded by pressure, de-pressuring the tubing after the concrete has cured, extracting the tubing thereby leaving vertical access tunnels in the foundation for testing.

Additional advantages of the invention are set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

A better understanding of the present invention can be obtained when the following detailed description of the disclosed embodiments is considered in conjunction with the following drawings in which:

FIG. 1 is a perspective view of a structural cage with attached removable tubing prior to insertion into an excavated hole;

FIG. 2 is a top view of a structural cage with attached removable tubing prior to insertion into an excavated hole;

FIG. 3 is a perspective view of a structural cage with attached removable tubing after insertion into an excavated hole;

FIG. 4 is a perspective view of a structural cage with attached removable tubing after insertion into the excavated hole with the removable tubing being pressurized during and after concrete fill;

FIG. 5 is a top view of a structural cage with attached removable tubing before pressurization of the removable tubing;

FIG. 6 is top view of a structural cage with attached removable tubing after pressurization of the removable tubing;

FIG. 7 is a perspective view of the structural cage with attached removable tubing embedded in cured concrete in the excavated hole with the tubing pressurized;

FIG. 8 is a perspective view of the structural cage with attached removable tubing embedded in cured concrete in the excavated hole after the tubing is de-pressurized;

FIG. 9 is a perspective view of the structural cage embedded in cured concrete in the excavated hole with the depressurized tubing being removed; and

FIG. 10 is a perspective view of the completed foundation with structural cage embedded in cured concrete with vertical access cores

### DETAILED DESCRIPTION OF THE INVENTION

The present invention is a system and a method for providing preformed vertical access tunnels for testing foundation structures. The system comprises a structural cage having generally vertical structural members as well as confining structural members, and tubing running generally vertically and removably attached to the structural cage. The method of providing preformed vertical access tunnels for testing foundation structures comprises providing the system and an excavated hole, introducing the system into the excavated hole, pour concrete into the hole to encase structural cage, pressuring the removable tubing during or after filling the hole with concrete thereby expanding the diameter of the tubing, allow the concrete to cure while the tubing remains expanded by pressure, de-pressuring the tubing after the concrete has cured, extracting the tubing thereby leaving vertical access tunnels in the foundation for testing.

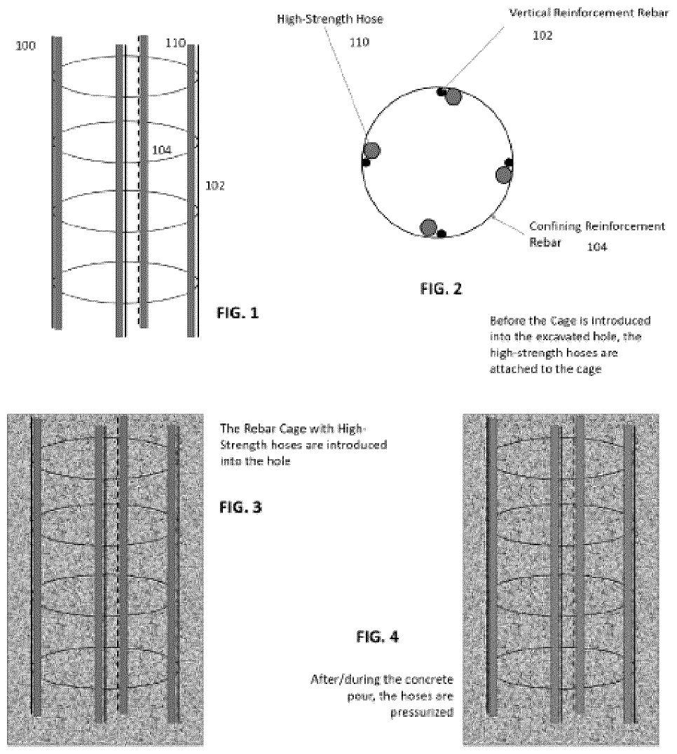

As shown in FIGS. 1 and 2, in preferred embodiments, the structural cage comprises a cage 100 with bars, preferably steel rebar, forming the generally vertical structural members 102 of the cage 100. The confining structural members 104 preferably surround the vertical structural members 102 and are securely attached to the vertical structural members 102. As will be recognized by a person of skill in the art, in the embodiment shown, the confining structural members 104 are generally circular, but other shapes or configurations such as square, hexagonal, etc. could be utilized depending upon the structural design and requirements. Similarly, while the confining structural members 104 surround the outside of the vertical structural members in the example shown, the confining structural members 104 could be on the interior or woven around the vertical structural members 102 provide they achieve the required structural support for the cage. While steel rebar is preferred in this embodiment to form the structural members, a person of skill in the art will recognize that other suitable materials of construction and variations in size can be utilized depending upon the structural design and requirements.

The example of FIGS. 1 and 2 shows four generally vertical members 102, but there could be any number of members 102 depending upon the structural design and requirements of the foundation structure being formed. Similarly, there are four confining members 104 shown, but the number of confining members can vary substantially depending upon the depth and size of the foundation being constructed, the size and materials of the bar used to construct the confining members 104, and other design considerations that will be recognized by one of skill in the art. As will be recognized by those of skill in the art, the structural cage is preferably preformed prior to insertion, but the invention broadly includes structural cages that are partially or fully assembled in bore.

Removable tubing 110 is attached running from near the top of the structural cage generally proximate to the surface and extends to near the bottom of the cage. Preferably, the tubing 110 is removably attached to the structural cage such that it is generally parallel with the vertical structural members. Attachment can be made by bans, rings, ties or other such means as will be known to those of skill in the art which allow the tubing 110 to be detached and removed at the end of the processes. The removal tubing can be flexible hose, capable of capable of expansion when under pressure. In preferred embodiments, removable tubing may comprise materials such as rubbers, plastics such as soft polyvinyl chloride, fluorinated ethylene propylene or other materials a person of skill in the art will recognize as having properties that allow expansion under fluid pressure and durability to sustain installation, concrete pouring and curing, and removal. In an alternative embodiment, the removable tubing comprises metal pipe or tubing coated to prevent adhesion of concrete.

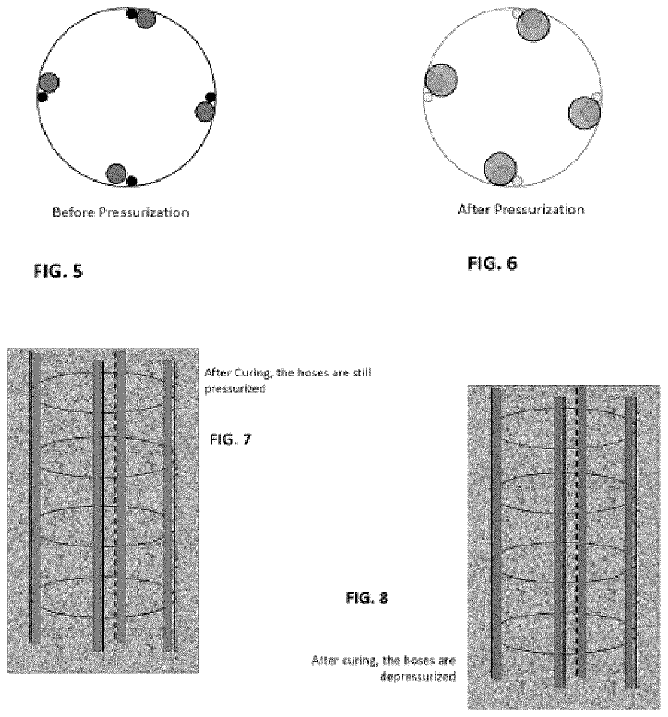

As shown in FIGS. 3 and 5, preferably the method of providing preformed vertical access tunnels for testing foundation structures comprises providing the structural cage system as described above and an excavated hole. The system is preferably introduced into the excavated hole prior to pouring concrete into the hole to encase structural cage. As will now be recognized by one of skill in the art, the excavated hole is then filled with an appropriate concrete mixture in accordance with the structural design and requirements for the foundation.

As shown in FIGS. 4 and 6, the removable tubing 110 is pressurized with fluids thereby expanding the diameter of the tubing 110. The removable tubing 110 can be pressurized before, during, or after concrete pouring is completed, but before the concrete is cured. As will be now be recognized by those of skill in the art, many fluids can be utilized to pressurize tubing 110. Preferably, water or a similar liquid is utilized, but gases such as air could be utilized depending upon the circumstances such as availability, structural design, and materials of construction.

As shown in FIGS. 7 and 8, the next steps in the process are to allow the concrete to cure while the tubing 110 remains expanded by pressure. Preferably, after curing then de-pressuring the tubing 110. As will be now be recognized by a person of skill, the pressurizing fluid can be removed from the tunnel or allowed to remain depending upon the fluid and the foundation particulars. In preferred embodiments, the removable tubing can be extracted from the tunnel and then reused for additional installations. In some embodiments, the removable tubing 100 is coated with a lubricant or other material such as is none to a person of skill in the art to minimize sticking to the surrounding concrete.

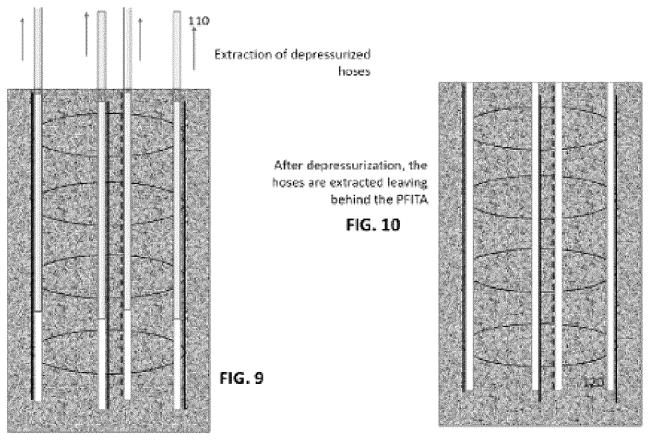

As shown in FIGS. 9 and 10, after the concrete has sufficiently cured, and the tubing 110 has been extracted from new foundation leaving vertical access tunnels 120 in the foundation are available for testing the foundation.

While the terms used herein are believed to be well-understood by one of ordinary skill in the art, definitions are set forth to facilitate explanation of certain of the presently-disclosed subject matter.

Following long-standing patent law convention, the terms “a”, “an”, and “the” refer to one or more when used in this application, including the claims. Thus, for example, reference to “a window” includes a plurality of such windows, and so forth.

Unless otherwise indicated, all numbers expressing quantities of elements, dimensions such as width and area, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of a dimension, area, percentage, etc., is meant to encompass variations of in some embodiments plus or minus 20%, in some embodiments plus or minus 10%, in some embodiments plus or minus 5%, in some embodiments plus or minus 1%, in some embodiments plus or minus 0.5%, and in some embodiments plus or minus 0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter. With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, S, C, and/or O” includes A, S, C, and O individually, but also includes any and all combinations and subcombinations of A, S, C, and O.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. The foregoing disclosure and description are illustrative and explanatory thereof, and various changes in the details of the illustrated apparatus and construction and method of operation may be made without departing from the spirit in scope of the invention which is described by the following claims.

We claim:

1. A method for providing preformed vertical access tunnels for testing foundation structures comprising the steps of:
providing a structural cage comprising generally vertical structural members and generally circular confining members, and an excavated hole;
removably attaching at least one vertical length of tubing to the structural cage generally parallel to the vertical structural members;
inserting the structural cage into the hole leaving the vertical structural members in a generally vertical position;
pouring concrete into and filling the excavated hole surrounding the structural cage;
pressurizing the tubing with a fluid before the concrete has cured thereby allowing the tubing to expand in diameter;
depressurizing the tubing; and
extracting the tubing thereby leaving a generally vertical access tunnel in the foundation.

2. The method of claim 1 wherein the pressurizing the tubing step comprises pressurizing the tubing with a liquid.

3. The method of claim 1 wherein the pressurizing the tubing step comprises pressurizing the tubing with a gas.

4. The method of claim 1 wherein the pressurizing the tubing step comprises pressurizing the tubing with water.

5. The method of claim 1 wherein the tubing is removably attached using bans.

6. The method of claim 1 wherein the pressurizing the tubing step occurs during the pouring concrete step.

7. The method of claim 1 wherein the pressurizing the tubing step occurs before the pouring concrete step.

8. The method of claim 1 wherein the fluid used for pressurizing is at least partially removed from the generally vertical access tunnel in the foundation.

9. The method of claim 1 wherein a plurality of vertical lengths of tubing are utilized in the removably attaching step and the remaining steps are utilized to leave a plurality of generally vertical access tunnels in the foundation.

10. The method of claim 1 wherein the at least one vertical length of tubing is coated with a material to prevent sticking to concrete.

11. The method of claim 1 wherein the at least one vertical length of tubing comprises plastic tubing.

12. The method of claim 1 wherein after the pressurizing the tubing step, the tubing has an inside outside diameter of between about 38 mm and 76 mm.

13. The method of claim 1 wherein the structural cage is preformed and the removably attaching step occurs prior to inserting the structural cage into the excavation hole.

14. A system for providing preformed vertical access tunnels for testing foundation structures comprising:
a structural cage comprising generally vertical structural members and generally circular confining members, and an excavated hole; and
at least one vertical length of tubing removably attached to the structural cage generally parallel to the vertical structural member.

15. The system of claim 14 wherein said one vertical length of tubing comprises plastic tubing.

16. The system of claim 14 wherein said at least one vertical length of tubing is removably attached using banding.

17. The system of claim 14 wherein said at least one vertical length of tubing is coated to prevent sticking to concrete.

18. The system of claim 14 wherein there are plurality of vertical lengths of tubing removably attached to the structural cage generally parallel to the vertical structural member.
